(19) United States
(12) Patent Application Publication
(10) Pub. No.: US 2015/0374348 A1
(43) Pub. Date: Dec. 31, 2015

(54) Use Of Vibration For EUS-FNA Tissue Acquisition

(71) Applicant: Boston Scientific Scimed, Inc., Maple Grove, MN (US)

(72) Inventor: John Allen HINGSTON, Framingham, MA (US)

(21) Appl. No.: 14/746,145

(22) Filed: Jun. 22, 2015

Related U.S. Application Data
(60) Provisional application No. 62/017,629, filed on Jun. 26, 2014.

(51) Int. Cl. A61B 10/04; A61B 1/018; A61B 10/02; A61B 1/12; A61B 17/32; A61B 8/08; A61B 8/12

(52) U.S. Cl. CPC A61B 10/04; A61B 8/0841; A61B 8/481; A61B 1/018; A61B 8/12; A61B 1/128; A61B 17/320068; A61B 10/0283; A61B 2017/00212

(57) ABSTRACT

A needle includes an elongate body extending longitudinally from a proximal end to a distal tip and including a lumen extending therethrough and a piezoelectric element positioned along a distal portion of the body so that, when a potential difference is applied thereto, the piezoelectric element expands and contracts to vibrate the distal tip of the elongate body.

### PRIORITY CLAIM

The present disclosure claims priority to U.S. Provisional Patent Application Ser. No. 62/017,629 filed Jun. 26, 2014 which is incorporated herewith by reference

### BACKGROUND

Needle biopsy procedures may be used to diagnose and to stage a disease. In particular, in endoscopic ultrasound-guided fine needle aspiration (EUS-FNA), the needle is advanced under ultrasound guidance so that the physician is able to visualize a position of the needle in relation to the target tissue. A distal end of the needle is then inserted into the target tissue to collect a sample of the tissue in a lumen thereof. Thus, EUS-FNA ensures that the correct tissue is sampled while minimizing risk to the patient. Although EUS-FNA is a highly sensitive and specific procedure, it is often difficult to acquire a suitable sample under certain clinical situations. The more cells or tissue that can be acquired, the greater the potential for a definitive diagnosis. Larger gauge needles, however, may be difficult to pass through tortuous anatomy. In other implementations, the samples from these needles may include more blood, making it more difficult to obtain a diagnosis.

### SUMMARY

The present disclosure is directed to a needle, comprising an elongate body extending longitudinally from a proximal end to a distal tip and including a lumen extending therethrough and a piezoelectric element positioned along a distal portion of the body so that, when a potential difference is applied thereto, the piezoelectric element expands and contracts to vibrate the distal tip of the elongate body.

In an embodiment, the needle may further comprise a first conductor extending from a distal end connected to a distal end of the piezoelectric element to a proximal end connected to an electrical energy source and a second conductor extending from a distal end connected to a proximal end of the piezoelectric element to a proximal end connected to the electrical energy source, one of the first and second conductors delivering a negative current to the piezoelectric element and the other of the first and second conductors delivering a positive current to the piezoelectric element.

In an embodiment, a first end of the piezoelectric element may be a potential ground and a second end of the piezoelectric element may form a varying potential difference varying between a negative and positive potential difference relative to the first end.

In an embodiment, the piezoelectric element may form a portion of the length of the elongate body and may include a lumen extending therethrough, the lumen being aligned with and having a diameter substantially corresponding to that of the lumen of the elongate body.

In an embodiment, the needle may further comprise a vacuum source connected to the proximal end of the elongate body for applying a suction force therethrough.

In an embodiment, the needle may further comprise an echogenic pattern along an exterior surface of the elongate body.

In an embodiment, the piezoelectric element may include a metal coating applied over an exterior surface thereof.

In an embodiment, the metal material may be one of chromium cobalt and nitinol.

In an embodiment, the coating may include an echogenic pattern formed thereon.

In an embodiment, an interior surface of the distal tip may be coated with a thermal conductor.

In an embodiment, the thermal conductor may be one of copper and silver.

In an embodiment, the electrical source may include an actuator for hands-free actuation thereof

The present disclosure is also directed to a device for collecting a tissue sample, comprising a needle extending longitudinally from a proximal end to a distal tip, the needle including a lumen extending therethrough and a piezoelectric element positioned along a distal portion thereof, a first conductor extending from a distal end connected to a distal end of the piezoelectric element to a proximal end connected to an electrical energy source, and a second conductor extending from a distal end connected to a proximal end of the piezoelectric element to a proximal end connected to the electrical energy source, one of the first and second conductors delivering a negative current to the piezoelectric element and the other of the first and second conductors delivering a positive current to the piezoelectric element such that, when a potential difference is applied thereto, the piezoelectric element expands and contracts to vibrate the distal tip of the elongate body.

In an embodiment, the piezoelectric element may be positioned proximally of the distal tip so that only the distal tip vibrates.

In an embodiment, the first and second conductors may be embedded in a wall of the needle.

The present disclosure is also directed to a method for collecting a tissue sample, comprising positioning a needle through a working channel of an endoscope to a target tissue within a patient body, inserting a distal tip of the needle into the target tissue to receive a core tissue sample in a lumen of the needle, and applying a potential differential to a piezoelectric element positioned along a distal portion of the needle to actuate a vibration of the distal tip, the vibration of the needle cutting the core tissue sample from a surrounding portion of the target tissue.

### BRIEF DESCRIPTION OF THE DRAWINGS

FIG. 1 shows a schematic side view of a device according to an exemplary embodiment of the present disclosure; and

FIG. 2 shows a transparent perspective view of the device of FIG. 1.

### DETAILED DESCRIPTION

The present disclosure may be further understood with reference to the following description and the appended drawings, wherein like elements are referred to with the same reference numerals. The present disclosure related to devices for obtaining tissue samples and, in particular, EUS-FNA devices. Exemplary embodiments of the present disclosure describe an EUA-FNA device comprising a distal tip vibrating via a potential difference applied to a piezoelectric element positioned along a portion of the device to aid in the cutting of tissue to acquire a core sample. It should be noted that the terms “proximal” and “distal” as used herein, are intended to refer to a direction toward (proximal) and away from (distal) a user of the device.

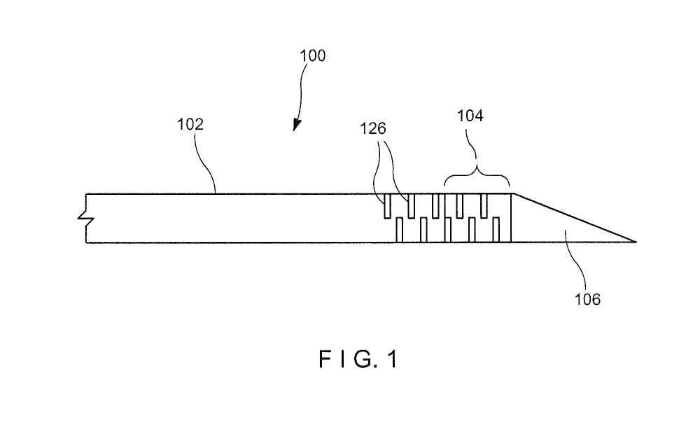

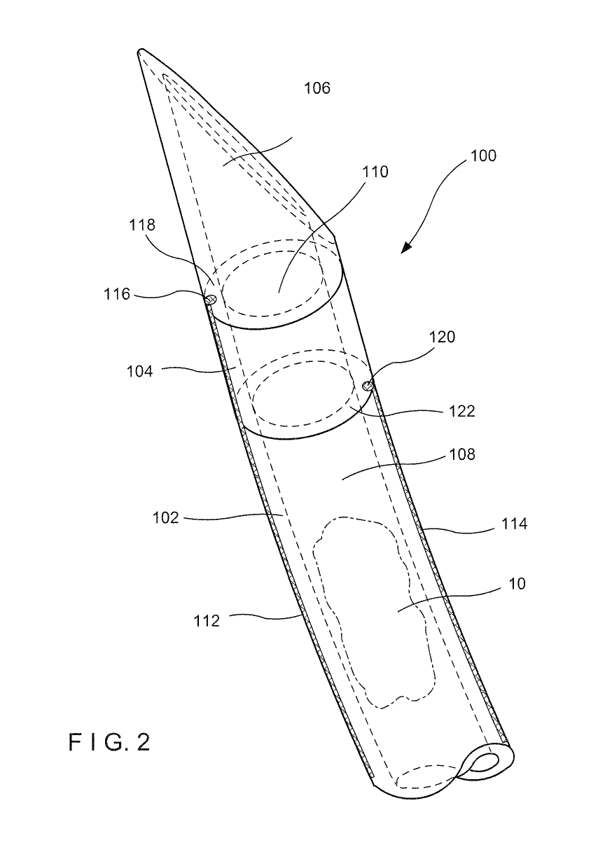

As shown in FIGS. 1 and 2, a needle 100 according to an exemplary embodiment of the present disclosure comprises an elongate body 102 and a piezoelectric element 104 positioned along a portion of the elongate body 102. The piezoelectric element 104 converts electrical energy to mechanical energy such that a distal tip 106 of the elongate body 102 vibrates to aid in cutting tissue and/or cauterizing to acquire a core tissue sample 10. Methods utilizing current existing EUS-FNA needles involve repeatedly inserting the needle into the tissue to separate the tissue sample from the surrounding tissue. In some cases, however, this methodology may produce a tissue sample or histology with significant blood contamination, preventing an accurate diagnosis. The vibrating distal tip 106 minimizes trauma and, therefore, minimizes blood contamination, enabling the collection of a better core sample.

The elongate body 102 extends longitudinally from a proximal end (not shown) to the distal tip 106 and includes a lumen 108 extending therethrough. The tip 106 may be tapered and/or sharpened to facilitate piercing tissue or surfaces for the collection of a tissue sample 10 in the lumen 108. A distal edge of the tip 106 may also be sharpened to facilitate a cutting of the core tissue sample 10 from the surrounding tissue as the distal tip 106 vibrates. The elongate body 102 is sized and shaped to be passed through a working channel of an endoscope and is formed of a flexible metal material which permits the needle 100 to be passed through tortuous paths in the body—e.g., following the path of a body lumen through which the endoscope is inserted via a naturally occurring body orifice. The elongate body 102 may further include echogenicity enhancing features 126 such as, for example, grooves, dimples or recesses along an exterior surface of the elongate body 102 to facilitate imaging of the needle as would be understood by those skilled in the art. As would be understood by those skilled in the art, the lumen 108 may receive a stylet (not shown) therein so that, during insertion, the stylet may be moved to a distal end of the lumen 108 to seal the lumen 108 preventing non-targeted tissue from entering the lumen 108. Furthermore, a proximal end of the elongate body 102 may be coupled to a vacuum source to assist in drawing target tissue into the lumen 108.

The piezoelectric element 104 is mounted along a portion of the elongate body 102 and is electrically connected to a proximal portion (not shown) of the needle 100 via first and second conductors 112, 114 which may be mounted at or within a wall of the elongate body 102. The proximal portion of the needle 100 includes source of electric energy (or a coupling for attachment to a source of electrical energy) for delivering a driving voltage to the piezoelectric element 104. In an exemplary embodiment, the piezoelectric element 104 is mounted at a distal portion of the elongate body 102, proximal of the tapered distal tip 106. In particular, the piezoelectric element 104 may be positioned along the elongate body 102 so that only the distal tip 107 vibrates. However, it will be understood by those of skill in the art that the piezoelectric element 104 may be positioned along any portion of the elongate body 102. Alternatively, the piezoelectric element 104 and tip 106 may be merged to form a single element with the vibration element and the tip combined. A diameter of the piezoelectric element 104 is preferably selected to be similar to that of the elongate body 102 such that mounting the piezoelectric element along a portion thereof does not interfere with a functioning of the needle 100. In particular, the piezoelectric element 104 may be substantially tubular including a lumen 110 extending therethrough, the lumen 110 of the piezoelectric element 110 being aligned with the lumen 108 of the elongate body 102. An outer diameter of the piezoelectric element 104 may be substantially similar an outer diameter of the elongate body 102 and an inner diameter may be substantially similar to an inner diameter of the lumen 108.

The first conductor 112 extends from a distal end 116 connected to a distal end 118 of the piezoelectric element 104 to a proximal end connected to the electrical energy source. The second conductor 114 extends from a distal end 120 connected to a proximal end 122 of the piezoelectric element 114 to a proximal end connected to the electrical energy source. The first conductor 112 may apply a negative current to the distal end 116 of the piezoelectric element 104 with a portion of the metal nail body extending distally of the piezoelectric element 104 (e.g., the distal tip 106) acting as the negative potential difference terminal and the second conductor 114 may apply a positive current to the proximal end of the piezoelectric element 104 with a portion of the elongate body 102 extending proximally of the piezoelectric element 104 acting as the positive potential difference terminal. According to an alternate embodiment, a portion of the lumen 108 may include or be formed of an electrically conducting material such as, for example, stainless steel, so that first and/or second conductors 112, 114 are not required. The piezoelectric element 104 expands and contracts to vibrate the distal tip 106 when the potential difference (voltage)/alternating current is applied across the piezoelectric element 104. The frequency of the alternating current dictates the frequency at which the distal tip 106 vibrates. A desired length of the piezoelectric element 104 (i.e., a distance between the proximal and distal ends 122, 118 of the piezoelectric element 104) may be a function of the desired amplitude of the vibration. In an exemplary embodiment of the present disclosure, however, the length of the piezoelectric element 104 may range from a submillimeter to tens of millimeters. Although the first conductor 112 is described as delivering a negative current while the second conductor 114 delivers a positive current, it will be understood by those of skill in the art that the first conductor 112 may alternatively deliver a positive current while the second conductor 114 delivers a negative current. Alternatively, a first end (e.g., one of the proximal and distal ends 122, 120) of the piezoelectric electrode 104 may be a potential ground while a second end (e.g., the other of the proximal and distal ends 122, 120) of the piezoelectric electrode 104 may form a varying potential difference swinging between negative and positive potential differences relative to the first end.

It is noted that the needle 100 is primarily intended to cut the core tissue sample 10 from the tissue and is not intended to burn or harm the tissue in any way. An amplitude and frequency of the vibration should be sufficient to maximize the cutting action of the distal tip 106 while preventing the tissue from being “burned.” Some amount of coagulation, however, may be desired to minimize the amount of blood in the core tissue sample 10. Thus, it is respectfully submitted that an ideal vibration frequency for the needle 100 would be much lower than for a device intended to coagulate tissue to stop bleeding. In an exemplary embodiment a range of frequency may be between 1 Hz and 100,000 Hz (100 kHz), more particularly between 100 Hz and 10,000 (10 kHz), and even more particularly between 900 Hz and 1,100 Hz (1.1 kHz) or about 1000 Hz (1 kHz). In addition, bursts of power may be utilized to minimize the amount of heat generated. It is also noted that once the core tissue sample 10 is cut from the surrounding tissue, it may be drawn into the lumen 108 of the elongate body 102 proximally of the vibrating distal tip 106 via, for example, a suction force, such that heat from the distal tip 106 does not affect the collected core sample 10. Further, the needle 100 may be designed to carry away heat from the distal tip 106 by, for example, coating an inner surface of the distal tip 106 (e.g., a surface of the lumen 108 at the distal tip 106) with a thermal conductor such as copper or silver. In addition, the amplitude of the vibration may be varied to minimize heat generation. This may be controlled by varying a degree of the potential difference applied.

As described above, the needle 100 may further include echogenic patterns 126 such as protrusions, grooves or recesses along a portion of the elongate body 102 to aid in ultrasound visualization. A length of the piezoelectric element 104 may be selected to minimize any effect on the echogenic pattern. In another embodiment, an exterior surface of the piezoelectric element 104 may be coated with a metal material such as cobalt chromium or nitinol so that the echogenic pattern 126 may be continued along the exterior of the piezoelectric element 104.

According to an exemplary surgical technique using the needle 100, the needle 100 is inserted to a target area within a patient body through a working channel of an endoscope via ultrasound guidance. The distal tip 106 of the elongate body 102 is then inserted into the target tissue to collect the core tissue sample 10 within the lumen 108. A user of the needle 100 then actuates the vibration feature (e.g., by pushing a button, stepping on a pedal or other hands-free actuation) to deliver alternating current to the piezoelectric element 104, causing the distal tip 106 to vibrate. The vibration of the distal tip 106 aids in cutting the core tissue sample 10 from the surrounding tissue and may help cauterize the tissue reducing the possibility of blood contamination. A vacuum may be applied through the lumen 108 to draw the core tissue sample 10 into the lumen 108 and away from the piezoelectric electrode 104. Once the core tissue sample 10 has been collected, as desired, the vibration may be stopped and the needle 100 removed from the patient body. Pressure may then be applied through the lumen 108 to remove the core sample 10.

It will be apparent to those skilled in the art that variations can be made in the structure and methodology of the present disclosure, without departing from the scope of the disclosure. Thus, it is intended that the present disclosure cover the modifications and variations of this disclosure provided that they come within the scope of the appended claims and their equivalents.

What is claimed is:

1. A needle, comprising:
an elongate body extending longitudinally from a proximal end to a distal tip and including a lumen extending therethrough; and
a piezoelectric element positioned along a distal portion of the elongate body so that, when a potential difference is applied thereto, the piezoelectric element expands and contracts to vibrate the distal tip of the elongate body.

2. The needle of claim 1, further comprising a first conductor extending from a distal end connected to a distal end of the piezoelectric element to a proximal end connected to an electrical energy source and a second conductor extending from a distal end connected to a proximal end of the piezoelectric element to a proximal end connected to the electrical energy source, one of the first and second conductors delivering a negative current to the piezoelectric element and the other of the first and second conductors delivering a positive current to the piezoelectric element.

3. The needle of claim 1, wherein a first end of the piezoelectric element is a potential ground and a second end of the piezoelectric element forms a varying potential difference varying between a negative and positive potential difference relative to the first end.

4. The needle of claim 1, wherein the piezoelectric element forms a portion of the length of the elongate body and includes a lumen extending therethrough, the lumen being aligned with and having a diameter substantially corresponding to that of the lumen of the elongate body.

5. The needle of claim 1, further comprising a vacuum source connected to the proximal end of the elongate body for applying a suction force therethrough.

6. The needle of claim 1, further comprising an echogenic pattern along an exterior surface of the elongate body.

7. The needle of claim 1, wherein the piezoelectric element includes a metal coating applied over an exterior surface thereof.

8. The needle of claim 7, wherein the metal material is one of chromium cobalt and nitinol.

9. The needle of claim 7, wherein the metal coating includes an echogenic pattern formed thereon.

10. The needle of claim 1, wherein an interior surface of the distal tip is coated with a thermal conductor.

11. The needle of claim 10, wherein the thermal conductor is one of copper and silver.

12. The needle of claim 2, wherein the electrical source includes an actuator for hands-free actuation thereof.

13. A device for collecting a tissue sample, comprising:
a needle extending longitudinally from a proximal end to a distal tip, the needle including a lumen extending therethrough and a piezoelectric element positioned along a distal portion thereof;
a first conductor extending from a distal end connected to a distal end of the piezoelectric element to a proximal end connected to an electrical energy source; and
a second conductor extending from a distal end connected to a proximal end of the piezoelectric element to a proximal end connected to the electrical energy source, one of the first and second conductors delivering a negative current to the piezoelectric element and the other of the first and second conductors delivering a positive current to the piezoelectric element such that, when a potential difference is applied thereto, the piezoelectric element expands and contracts to vibrate the distal tip of the elongate body.

14. The device of claim 13, wherein the piezoelectric element is positioned proximally of the distal tip so that only the distal tip vibrates.

15. The device of claim 13, wherein the first and second conductors are embedded in a wall of the needle.

16. A method for collecting a tissue sample, comprising:
positioning a needle through a working channel of an endoscope to a target tissue within a patient body;
inserting a distal tip of the needle into the target tissue to receive a core tissue sample in a lumen of the needle; and
applying a potential differential to a piezoelectric element positioned along a distal portion of the needle to actuate a vibration of the distal tip, the vibration of the needle cutting the core tissue sample from a surrounding portion of the target tissue.

17. The method of claim 16, further comprising applying a suction force through the lumen of the needle to draw the core tissue sample into the lumen.

18. The method of claim 16, wherein an interior surface of the distal tip is coated with a thermal conductor to conduct away heat generated by the vibration of the distal tip.

19. The method of claim 16, wherein the needle includes an echogenic pattern along an exterior surface thereof to aid in ultrasound visualization.

20. The method of claim 16, wherein the piezoelectric element is coated with one of chromium cobalt and nitinol, the echogenic pattern continuing along the coated exterior surface of the piezoelectric element.
